(19) United States
(12) Patent Application Publication
(10) Pub. No.: US 2026/0183314 A1
(43) Pub. Date: Jul. 2, 2026

(54) COMPOSITION FOR SUPPRESSING SEBUM COMPRISING CANNABIDIOL AND SALICYLIC ACID

(71) Applicant: Kolmar Korea Co., Ltd., Sejong-si (KR)

(72) Inventors: Yoon Gyung KWON, Seoul (KR); Byoung Jun Park, Seoul (KR)

(21) Appl. No.: 19/431,959

(22) Filed: Dec. 23, 2025

(30) Foreign Application Priority Data
Dec. 27, 2024 (KR) 10-2024-0199141

(51) Int. Cl. A61K 31/60; A61K 8/34; A61K 8/368; A61K 31/00; A61P 17/08; A61Q 19/00

(52) U.S. Cl. CPC A61K 31/60; A61K 8/347; A61K 8/368; A61K 31/658; A61P 17/08; A61Q 19/008; A61K 2800/592

(57) ABSTRACT

A composition for suppressing sebum synthesis, the composition including cannabidiol and salicylic acid.

### CROSS REFERENCE TO RELATED APPLICATION

This application claims priority from and the benefit of Korean Patent Application No. 10-2024-0199141, filed on Dec. 27, 2024, which is hereby incorporated by reference for all purposes as if fully set forth herein.

### BACKGROUND

### Field

Embodiments of the invention relate to a composition for suppressing sebum including cannabidiol and salicylic acid.

### Discussion of the Background

Acne is an inflammatory skin disease that affects the sebaceous glands of the skin and refers to folliculitis that mainly occurs on the human face. Generally, acne is known to be caused by a complex interaction of increased sebum secretion due to hormones, excessive keratinization, bacterial proliferation, genetic factors, and hair follicle reactivity.

Acne can be treated with topical medications, oral medications, and surgical procedures. However, since these treatments use retinoids and hormones, there are problems of side effects such as skin rashes, dry skin, allergies, and the suppression of epidermal growth when taken in excess.

To solve these problems, ongoing efforts are being made to develop naturally derived substances that can treat acne.

The above information disclosed in this Background section is only for understanding of the background of the inventive concepts, and, therefore, it may contain information that does not constitute prior art.

### SUMMARY

The embodiments of the invention are directed to providing a composition for suppressing sebum including cannabidiol and salicylic acid.

The technical objects of the invention are not limited to the technical object mentioned above, and other technical objects not mentioned herein will be clearly understood by those of ordinary skill in the art from the following description.

One aspect of the invention provides a composition for suppressing sebum. The composition for suppressing sebum includes cannabidiol and salicylic acid.

Additional features of the inventive concepts will be set forth in the description which follows, and in part will be apparent from the description, or may be learned by practice of the inventive concepts.

In addition, the weight ratio of the cannabidiol and the salicylic acid may be 1:0.8 to 3.

In addition, the composition for suppressing sebum may be present at a concentration of less than 5 μg/mL.

In addition, at a concentration of 1 μg/mL, the composition for suppressing sebum may reduce sebum production in sebocytes by 27% or more compared to the control containing 50 μM arachidonic acid, as measured by a lipogenesis assay.

In addition, at a concentration of 2.5 μg/mL, the composition for suppressing sebum may reduce sebum production in sebocytes by 30% or more compared to the control containing 50 μM arachidonic acid, as measured by a lipogenesis assay.

Another aspect of the invention provides a cosmetic composition including the composition for suppressing sebum.

Still another aspect of the invention provides an external composition for skin, which includes the composition for suppressing sebum.

Yet another aspect of the invention provides a pharmaceutical composition for treating or preventing an inflammatory disease, which includes the composition for suppressing sebum.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention, and together with the description serve to explain the inventive concepts.

FIG. 1 is a graph illustrating the results of a cytotoxic evaluation for compositions for suppressing sebum according to examples and comparative examples of the invention.

FIG. 2 is a graph illustrating the results of a sebum suppression efficacy evaluation of compositions for suppressing sebum according to examples and comparative examples of the invention.

### DETAILED DESCRIPTION

In the following description, for the purposes of explanation, numerous specific details are set forth in order to provide a thorough understanding of various embodiments or implementations of the invention. As used herein “embodiments” and “implementations” are interchangeable words that are non-limiting examples of devices or methods employing one or more of the inventive concepts disclosed herein. It is apparent, however, that various embodiments may be practiced without these specific details or with one or more equivalent arrangements. In other instances, well-known structures and devices are shown in block diagram form in order to avoid unnecessarily obscuring various embodiments. Further, various embodiments may be different, but do not have to be exclusive. For example, specific shapes, configurations, and characteristics of an embodiment may be used or implemented in another embodiment without departing from the inventive concepts.

Unless otherwise specified, the illustrated embodiments are to be understood as providing features of varying detail of some ways in which the inventive concepts may be implemented in practice. Therefore, unless otherwise specified, the features, components, modules, layers, films, panels, regions, and/or aspects, etc. (hereinafter individually or collectively referred to as “elements”), of the various embodiments may be otherwise combined, separated, interchanged, and/or rearranged without departing from the inventive concepts.

The use of cross-hatching and/or shading in the accompanying drawings is generally provided to clarify boundaries between adjacent elements. As such, neither the presence nor the absence of cross-hatching or shading conveys or indicates any preference or requirement for particular materials, material properties, dimensions, proportions, commonalities between illustrated elements, and/or any other characteristic, attribute, property, etc., of the elements, unless specified. Further, in the accompanying drawings, the size and relative sizes of elements may be exaggerated for clarity and/or descriptive purposes. When an embodiment may be implemented differently, a specific process order may be performed differently from the described order. For example, two consecutively described processes may be performed substantially at the same time or performed in an order opposite to the described order. Also, like reference numerals denote like elements.

When an element, such as a layer, is referred to as being “on,” “connected to,” or “coupled to” another element or layer, it may be directly on, connected to, or coupled to the other element or layer or intervening elements or layers may be present. When, however, an element or layer is referred to as being “directly on,” “directly connected to,” or “directly coupled to” another element or layer, there are no intervening elements or layers present. To this end, the term “connected” may refer to physical, electrical, and/or fluid connection, with or without intervening elements. Further, the D1-axis, the D2-axis, and the D3-axis are not limited to three axes of a rectangular coordinate system, such as the x, y, and z-axes, and may be interpreted in a broader sense. For example, the D1-axis, the D2-axis, and the D3-axis may be perpendicular to one another, or may represent different directions that are not perpendicular to one another. For the purposes of this disclosure, “at least one of X, Y, and Z” and “at least one selected from the group consisting of X, Y, and Z” may be construed as X only, Y only, Z only, or any combination of two or more of X, Y, and Z, such as, for instance, XYZ, XYY, YZ, and ZZ. As used herein, the term “and/or” includes any and all combinations of one or more of the associated listed items.

Although the terms “first,” “second,” etc. may be used herein to describe various types of elements, these elements should not be limited by these terms. These terms are used to distinguish one element from another element. Thus, a first element discussed below could be termed a second element without departing from the teachings of the disclosure.

Spatially relative terms, such as “beneath,” “below,” “under,” “lower,” “above,” “upper,” “over,” “higher,” “side” (e.g., as in “sidewall”), and the like, may be used herein for descriptive purposes, and, thereby, to describe one elements relationship to another element(s) as illustrated in the drawings. Spatially relative terms are intended to encompass different orientations of an apparatus in use, operation, and/or manufacture in addition to the orientation depicted in the drawings. For example, if the apparatus in the drawings is turned over, elements described as “below” or “beneath” other elements or features would then be oriented “above” the other elements or features. Thus, the exemplary term “below” can encompass both an orientation of above and below. Furthermore, the apparatus may be otherwise oriented (e.g., rotated 90 degrees or at other orientations), and, as such, the spatially relative descriptors used herein interpreted accordingly.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof. It is also noted that, as used herein, the terms “substantially,” “about,” and other similar terms, are used as terms of approximation and not as terms of degree, and, as such, are utilized to account for inherent deviations in measured, calculated, and/or provided values that would be recognized by one of ordinary skill in the art.

Various embodiments are described herein with reference to sectional and/or exploded illustrations that are schematic illustrations of idealized embodiments and/or intermediate structures. As such, variations from the shapes of the illustrations as a result, for example, of manufacturing techniques and/or tolerances, are to be expected. Thus, embodiments disclosed herein should not necessarily be construed as limited to the particular illustrated shapes of regions, but are to include deviations in shapes that result from, for instance, manufacturing. In this manner, regions illustrated in the drawings may be schematic in nature and the shapes of these regions may not reflect actual shapes of regions of a device and, as such, are not necessarily intended to be limiting.

As customary in the field, some embodiments are described and illustrated in the accompanying drawings in terms of functional blocks, units, and/or modules. Those skilled in the art will appreciate that these blocks, units, and/or modules are physically implemented by electronic (or optical) circuits, such as logic circuits, discrete components, microprocessors, hard-wired circuits, memory elements, wiring connections, and the like, which may be formed using semiconductor-based fabrication techniques or other manufacturing technologies. In the case of the blocks, units, and/or modules being implemented by microprocessors or other similar hardware, they may be programmed and controlled using software (e.g., microcode) to perform various functions discussed herein and may optionally be driven by firmware and/or software. It is also contemplated that each block, unit, and/or module may be implemented by dedicated hardware, or as a combination of dedicated hardware to perform some functions and a processor (e.g., one or more programmed microprocessors and associated circuitry) to perform other functions. Also, each block, unit, and/or module of some embodiments may be physically separated into two or more interacting and discrete blocks, units, and/or modules without departing from the scope of the inventive concepts. Further, the blocks, units, and/or modules of some embodiments may be physically combined into more complex blocks, units, and/or modules without departing from the scope of the inventive concepts.

Unless otherwise defined, all terms (including technical and scientific terms) used herein have the same meaning as commonly understood by one of ordinary skill in the art to which this disclosure is a part. Terms, such as those defined in commonly used dictionaries, should be interpreted as having a meaning that is consistent with their meaning in the context of the relevant art and should not be interpreted in an idealized or overly formal sense, unless expressly so defined herein.

Among the physical properties mentioned herein, when the measurement temperature affects the properties, unless otherwise specified, the properties are measured at room temperature and atmospheric pressure.

Unless otherwise specified among the properties mentioned herein, the corresponding properties may basically follow the SI unit system. For example, a weight may use kg units and a length may use m units.

As used herein, the term “room temperature” is a natural temperature that has not been heated or cooled, and may mean, for example, any one temperature within the range of 10 to 30° C., for example, approximately 15° C. or more, approximately 18° C. or more, approximately 20° C. or more, approximately 23° C. or more, approximately 27° C. or less, or 25° C. Unless specifically specified herein, the unit of temperature is Celsius (° C.).

As used herein, the term “relatively high temperature compared to room temperature” may mean a temperature higher than room temperature, for example, any one temperature within the range of higher than 30° C. to 50° C., for example, approximately 35° C. or more or approximately 40° C. or more, or approximately 45° C. or less.

Among the properties mentioned herein, when the measurement pressure affects the properties, unless otherwise specified, the properties are measured at atmospheric pressure. As used herein, the term “atmospheric pressure” refers to an atmospheric pressure that is normally in the range of approximately 700 to 800 mmHg as a natural pressure that is not pressurized and depressurized.

As used herein, the term “a to b” means within the range between a and b, including a and b. For example, including something at a to b parts by weight is the same as including within the range of a to b parts by weight.

Values expressed throughout the specification in a range format should be construed in a flexible manner to include not only numerical values explicitly referred to as the limits of such ranges, but also all individual numerical values or sub-ranges included within such ranges, as if each numerical value and sub-range were explicitly stated. For example, a range of “approximately 0.1% to approximately 5%” or “approximately 0.1% to 5%” should be construed to include not only approximately 0.1% to approximately 5%, but also individual values (e.g., 1%, 2%, 3%, and 4%) and sub-ranges (e.g., 0.1% to 0.5%, 1.1% to 2.2%, or 3.3% to 4.4%) within the stated range. Unless otherwise indicated, “approximately X to Y” has the same meaning as “approximately X to approximately Y.” Likewise, unless otherwise specified, “approximately X, Y, or Z” has the same meaning as “approximately X, approximately Y, or approximately Z.”

The term “approximately” or “about” used herein means that dimensions, sizes, formulations, parameters, shapes, and other quantities and characteristics may not be exact, and may be approximate and/or larger or smaller than the stated value, taking into account tolerances, conversion factors, rounding, measurement errors, and the like, and other factors known to those of ordinary skill in the art. The terms “approximately” or “about” generally apply to dimensions, sizes, formulations, parameters, shapes, or other quantities or characteristics described herein whether expressly stated or not. As used herein, the term “approximately” may allow a degree of variability in a given value or range, e.g., within 10%, 5%, or 1% of a stated value or range limit, and includes the exact value or range stated.

When methods of measuring or evaluating specific dimensions, size, formulation, parameter, shape, and other quantities and properties are not described herein, those of ordinary skill in the art can readily recognize conventional known measurement or evaluation methods, or can readily derive such measurement or evaluation methods by referring to techniques known prior to the filing date of the present specification.

As used herein, the term “substantially” refers to the majority or most such as approximately 50%, 60%, 70%, 80%, 90%, 95%, 96%, 97%, 98%, 99%, 99.5%, 99.9%, 99.99%, or at least approximately 99.999% or more, or 100%. As used herein, the term “substantially free of” may mean having nothing, or having a trivial amount of substance that does not affect the substantial properties of the composition containing that substance. Here, the trivial amount means that a composition contains a substance at approximately 0 to 5 wt %, or approximately 0 to 1 wt %, or approximately 5 wt % or less, or approximately 4.5 wt % or less, or approximately 4, 3.5, 3, 2.5, 2, 1.5, 1, 0.9, 0.8, 0.7, 0.6, 0.5, 0.4, 0.3, 0.2, 0.1, 0.01, or 0.001 wt % or less.

As used herein, the term “substantially” means 50% or more, 60% or more, 70% or more, 80% or more, or 90% or more.

The terminology used herein is for the purpose of describing embodiments and is not intended to limit or restrict the invention. Singular expressions include plural expressions unless the context clearly indicates otherwise. Additionally, ordinal numbers used herein (e.g., first, second, and the like.) are merely identifiers to distinguish one component from another.

Herein, when a part is said to be connected to another part, it includes not only cases where these parts are directly connected, but also cases where they are indirectly connected via another component therebetween. In addition, when a part includes another component, it means that it may further include another component, rather than excluding another component, unless otherwise specifically stated.

Moreover, in the invention, the term “or” is intended to mean an inclusive “or,” not an exclusive “or.” That is, unless otherwise specified or otherwise clear from the context, “X utilizes A or B” is intended to mean one of the natural inclusive substitutions. That is, when X utilizes A; X utilizes B; or X utilizes both A and B, “X utilizes A or B” can be applied to any of the above cases. It is also to be understood that the term “and/or” as used herein refers to and encompasses all possible combinations of one or more of the listed related components.

According to one embodiment of the invention, a composition for suppressing sebum, including cannabidiol and salicylic acid as active ingredients, is provided.

Cannabidiol is a type of cannabinoid, and known to provide various effects such as improving skin health, relieving pain, relieving anxiety, and reducing depression.

Cannabidiol may be extracted from the Cannabis sp plant, for example, Cannabis sativa, Cannabis chemovars, Cannabis indica, or Cannabis ruderalis.

Salicylic acid is a phenolic organic acid, known for its effectiveness in cleaning pores, removing dead skin cells, treating acne, reducing skin inflammation, and controlling sebum.

When the composition includes salicylic acid together with cannabidiol, due to the synergistic effect of cannabidiol and salicylic acid, the effect of suppressing sebum synthesis may increase, and the effect of improving an inflammatory disease such as acne or dermatitis may also be improved.

In one embodiment, the weight ratio of cannabidiol and salicylic acid may be 1:0.8 to 3. For example, the weight ratio of cannabidiol and salicylic acid may be 1:0.8 or more, 1:1 or more, 1:1.5 or more, 1:1.8 or more, 1:2 or more, 1:2.1 or more, 1:2.2 or more, 1:2.3 or more, 1:2.4 or more, or 1:2.5 or more. For example, the weight ratio of cannabidiol and salicylic acid may be 1:3 or less, 1:2.7 or less, 1:2.5 or less, 1:2.4 or less, 1:2.3 or less, 1:2.2 or less, 1:2.1 or less, 1:2 or less, 1:1.8 or less, or 1:1.5 or less.

In one embodiment, the weight ratio of cannabidiol and salicylic acid may be 1:1 to 2.5.

In one embodiment, the composition for suppressing sebum may be treated at a concentration of less than 5 μg/mL. For example, the composition for suppressing sebum may be treated at less than 5 μg/mL, 4.5 μg/mL or less, 4 μg/mL or less, 3 μg/mL or less, 2.5 μg/mL or less, 2 μg/mL or less, 1.5 μg/mL or less, or 1 μg/mL or less. For example, the composition for suppressing sebum may be treated at 0.0001 μg/mL or more, 0.001 μg/mL or more, 0.01 μg/mL or more, 0.1 μg/mL or more, 0.5 μg/mL or more, or 1 μg/mL or more.

When the weight ratio and/or treated concentration according to embodiments of the invention are satisfied, the composition may have advantages in safety to the extent that no cytotoxicity is shown and/or may have improved efficacy in sebum inhibition and/or improvement of inflammatory diseases such as acne and dermatitis.

In one embodiment, treatment with less than 5 μg/mL of the composition for suppressing sebum may reduce neutral lipid production in sebocytes by 25% or more compared to the control treated with 50 μM arachidonic acid, as measured by a lipogenesis assay. Arachidonic acid is a standard substance for inducing lipid production in sebocytes, and in the present specification, compared to the arachidonic acid-treated control, the degree (%) of reduction in neutral lipid production in sebocytes, measured by a lipogenesis assay, may be referred to as “sebum suppression efficacy.” For example, the degree of reduction in neutral lipid production may be calculated by setting the neutral lipid production of the arachidonic acid-treated control to 100%. For example, provided that the neutral lipid production of the arachidonic acid-treated control is 100 and the neutral lipid production of an experimental group is 75, the degree (%) of reduction in neutral lipid production in the experimental group may be 25%. For example, the sebum suppression efficacy of less than 5 μg/mL of the composition for suppressing sebum may be 25% or more, 25.5% or more, 26% or more, 26.5% or more, 27% or more, 27.5% or more, 28% or more, 28.5% or more, 29% or more, 29.5% or more, 30% or more, 30.5% or more, or 31% or more. For example, the sebum suppression efficacy of less than 5 μg/mL of the composition for suppressing sebum may be 50% or less, 40% or less, or 35% or less.

In one embodiment, treatment with 1 μg/mL of the composition for suppressing sebum may reduce neutral lipid production in sebocytes by 27% or more compared to the control treated with 50 μM arachidonic acid, as measured by a lipogenesis assay. That is, the sebum suppression efficacy of 1 μg/mL of the composition for suppressing sebum may be 27% or more. For example, the sebum suppression efficacy of 1 μg/mL of the composition for suppressing sebum may be 27% or more, 27.5% or more, 28% or more, 28.5% or more, or 29% or more. For example, the sebum suppression efficacy of 1 μg/mL of the composition for suppressing sebum may be 50% or less, 40% or less, or 35% or less.

In one embodiment, treatment with 2.5 μg/mL of the composition for suppressing sebum may reduce neutral lipid production in sebocytes by 30% or more compared to the control treated with 50 μM arachidonic acid, as measured by a lipogenesis assay. The sebum suppression efficacy of 2.5 μg/mL of the composition for suppressing sebum may be 30% or more. For example, the sebum suppression efficacy of 2.5 μg/mL of the composition for suppressing sebum may be 30% or more, 30.5% or more, or 31% or more. For example, the sebum suppression efficacy of 2.5 μg/mL of the composition for suppressing sebum may be 50% or less, 40% or less, or 35% or less.

In one embodiment, the composition for suppressing sebum may further include additives commonly used in the art. For example, the composition may further include a thickener, a binder, a denaturing agent, a flavoring agent, a moisturizer, a skin protectant, a pH adjuster, an isotonic agent, a preservative, an antiseptic, an antioxidant, or a fragrance. The content of the above-mentioned components may be easily selected by those of ordinary skill in the art without compromising the purpose and effect of the invention.

According to an embodiment of the invention, a cosmetic composition is provided. The cosmetic composition may include the above-described composition for suppressing sebum.

In the embodiment, the cosmetic composition may include the composition for suppressing sebum at a concentration of less than 5 μg/mL. For example, the concentration of the composition for suppressing sebum may be less than 5 μg/mL, 4.5 μg/mL or less, 4 μg/mL or less, 3 μg/mL or less, 2.5 μg/mL or less, 2 μg/mL or less, 1.5 μg/mL or less, or 1 μg/mL or less. For example, the concentration of the composition for suppressing sebum may be 0.0001 μg/mL or more, 0.001 μg/mL or more, 0.01 μg/mL or more, 0.1 μg/mL or more, 0.5 μg/mL or more, or 1 μg/mL or more.

In one embodiment, the cosmetic composition may be provided in a formulation commonly prepared in the art. For example, the cosmetic composition may be formulated into an oil dispersion, an aqueous dispersion, or an emulsion such as oil-in-water (O/W), water-in-oil (W/O), water-in-oil-in-water (W/O/W), or oil-in-water-in-oil (O/W/O), or a solubilized formulation. However, the present invention is not limited thereto, and the cosmetic composition may also be provided in a formulation, such as a solution, a suspension, an emulsion, a solid, a gel, an oil, a powder, a paste, or a foam aerosol.

In one embodiment, the cosmetic composition may be formulated into a variety of products. For example, the cosmetic composition may be formulated into hair cleansing products, such as shampoo, rinse, hair conditioner, hair gel, and the like. In addition, the cosmetic composition may be formulated into soap, cleaner, cleansing cream, or cleansing water, or into color cosmetics such as lipstick, lip balm, mascara, blush, shading, highlighter, makeup base, foundation, compact, concealer, skin cover, and the like. In addition, the cosmetic composition may be formulated into functional cosmetics such as nutrition cream, essence, and ampoules, or basic cosmetics such as toner, lotion, and the like. In addition, for example, the cosmetic composition may be formulated into a product that is attached to the skin, such as a pack, hydrogel slimming patch, or sprayed onto the skin, such as mist, aerosol, and the like.

In one embodiment, the cosmetic composition may further include an additive commonly used in the art to the extent that it does not adversely affect the effect of the invention. For example, the cosmetic composition may further include a thickener, a pH adjuster, an isotonic agent, a surfactant, a stabilizer, a preservative, an antiseptic, a bactericide, a humectant, a blocker, an antioxidant, an organic pigment, an inorganic pigment, a fragrance, or a vitamin. The content of the above-mentioned components may be easily selected by those of ordinary skill in the art without compromising the purpose and effect of the invention.

According to one embodiment of the invention, an external composition for skin is provided. The external composition for skin may include the above-described composition for suppressing sebum.

In one embodiment, the external composition for skin may include the composition for suppressing sebum at less than 5 μg/mL. For example, the concentration of the composition for suppressing sebum may be less than 5 μg/mL, 4.5 μg/mL or less, 4 μg/mL or less, 3 μg/mL or less, 2.5 μg/mL or less, 2 μg/mL or less, 1.5 μg/mL or less, or 1 μg/mL or less. For example, the concentration of the composition for suppressing sebum may be 0.0001 μg/mL or more, 0.001 μg/mL or more, 0.01 μg/mL or more, 0.1 μg/mL or more, 0.5 μg/mL or more, or 1 μg/mL or more.

The external composition for skin may mean a preparation that acts on the skin, among external preparations, and may include, but is not limited to, products such as pharmaceuticals and quasi-drugs. For example, the external composition for skin may take the form of a cream, a gel, a skin emulsifier, a patch, or a spray.

In one embodiment, the external composition for skin may appropriately include components commonly used in external skin preparations such as cosmetics or pharmaceuticals, for example, an aqueous component, an oily component, a powder component, an alcohol, a humectant, a thickener, a UV blocker, a whitening agent, a preservative, an antioxidant, a surfactant, a fragrance, a colorant, a fatty substance, a solubilizer, a concentrate, a gelling agent, an emollient, a foaming agent, an aromatic, a sequestrant, a chelating agent, a vitamin, various skin nutrients, or a combination thereof, as necessary. The content of the above-mentioned components may be easily selected by those of ordinary skill in the art without compromising the purpose and effect of the invention.

According to an embodiment of the invention, a pharmaceutical composition for preventing or treating an inflammatory disease is provided. The pharmaceutical composition may include the above-described composition for suppressing sebum. For example, inflammatory diseases may include dermatitis such as atopic dermatitis, seborrheic dermatitis, xerotic dermatitis, contact dermatitis, and nummular dermatitis, in addition to inflammation caused by acne, insect bites, and hives.

In one embodiment, the pharmaceutical composition may include the composition for suppressing sebum at less than 5 μg/mL. For example, the concentration of the composition for suppressing sebum may be less than 5 μg/mL, 4.5 μg/mL or less, 4 μg/mL or less, 3 μg/mL or less, 2.5 μg/mL or less, 2 μg/mL or less, 1.5 μg/mL or less, or 1 μg/mL or less. For example, the concentration of the composition for suppressing sebum may be 0.0001 μg/mL or more, 0.001 μg/mL or more, 0.01 μg/mL or more, 0.1 μg/mL or more, 0.5 μg/mL or more, or 1 μg/mL or more.

In one embodiment, the pharmaceutical composition may further include a pharmaceutically acceptable salt to the extent that it does not adversely affect the effect of the invention. Here, the “pharmaceutically acceptable salt” may be a salt that does not cause serious irritation to an organism to which the compound is administered and does not impair the biological activity and physical properties of the compound.

For example, the pharmaceutically acceptable salt may be formed from an inorganic acid, for example, hydrochloric acid, hydrobromic acid, sulfuric acid, nitric acid, phosphoric acid, or an organic acid such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, or N-acetylcysteine.

In addition, for example, the pharmaceutically acceptable salt may be prepared by adding an inorganic or organic base to a free acid. Salts derived from inorganic bases may include, but are not limited to, sodium, potassium, lithium, ammonium, calcium, and magnesium salts. Salts derived from organic bases may include, but are not limited to, salts of primary, secondary and tertiary amines, substituted amines including naturally occurring substituted amines, cyclic amines, and basic ion exchange resins such as isopropylamine, trimethylamine, diethylamine, triethylamine, tripropylamine, ethanolamine, lysine, arginine, N-ethylpiperidine, piperidine, polyimine resins, and the like.

In one embodiment, the pharmaceutical composition may further include a pharmaceutically acceptable carrier or additive to the extent that it does not adversely affect the effect of the invention. Here, “pharmaceutically acceptable carrier” is used commonly in formulations, and does not inhibit the activity of the active ingredient, does not have toxicity exceeding the tolerance limit of the subject of application or administration, without inhibiting the activity of the active ingredient, and facilitates the addition of the compound into cells or tissues.

In one embodiment, the pharmaceutical composition may be formulated as a solid or liquid formulation. For example, when formulated as a solid formulation, the pharmaceutical composition may further include a carrier, a flavoring agent, a binder, a preservative, a disintegrant, a lubricant, or a filler, and when formulated as a liquid formulation, the pharmaceutical composition may further include, but is not limited to, water, a solution such as a propylene glycol solution, a suspension, or an emulsion, and further include a colorant, a flavoring agent, a stabilizer, or a viscosifier.

In one embodiment, the solid formulation may include powder, granules, tablets, capsules, or suppositories. For example, the powder may be prepared by simply mixing a carrier such as lactose, starch, or microcrystalline cellulose with the active ingredient of the invention. The granules may be prepared by mixing the active ingredient of the invention with a carrier, and a binder such as polyvinylpyrrolidone or hydroxypropylcellulose, and then performing wet granulation using a solvent such as water, ethanol or isopropanol, or dry granulation using a compression. In addition, the tablets may be prepared by mixing the granules with a lubricant such as magnesium stearate and then tableting the resulting mixture using a tableting machine.

In one embodiment, the pharmaceutical composition may be administered orally, by injection (e.g., intramuscular injection, intraperitoneal injection, intravenous injection, infusion, subcutaneous injection, and implant), inhalation, nasal administration, vaginal administration, rectal administration, sublingual administration, transdermal administration, or topical administration, but the present invention is not limited thereto. In addition, the pharmaceutical composition of the invention may be formulated into a suitable dosage unit form including a pharmaceutically acceptable carrier, additive, or vehicle, which is conventionally used and non-toxic, depending on the administration route.

In one embodiment, the dosage of the pharmaceutical composition may be appropriately selected by those of ordinary skill in the art, although it depends on the patient's condition and body weight, the severity of a disease, a dosage form, and administration route and duration. The dosage form, and administration route and mode of the pharmaceutical composition may each be independent, are not particularly limited in their modes, and may follow any route and mode of administration that allows the pharmaceutical composition to reach the desired site. For example, the administration form of the pharmaceutical composition according to the embodiments of the invention may be used in the form of a pharmaceutically acceptable salt thereof, and may be used alone or in combination with other pharmaceutically active compounds, as well as in suitable combinations. In addition, for example, the administration mode of the pharmaceutical composition according to the embodiment of the invention may be intravenous administration, intraperitoneal administration, intramuscular administration, transdermal administration, or subcutaneous administration, as well as methods such as applying, spraying, or inhaling the composition to the disease site, but the present invention is not limited thereto.

Hereinafter, the following examples are provided to explain the embodiments of the invention in further detail, but the embodiment is not limited thereto.

### Preparation Example: Preparation of Examples 1 to 3 and Comparative Examples 1 to 5

Compositions containing cannabidiol and/or salicylic acid of Examples 1 to 3 and Comparative Examples 1 to 5 were prepared according to the weight ratios listed in Table 1 while the total weight of cannabidiol and salicylic acid contained in the composition remained the same. For example, in an example or comparative example in Table 1, when cannabidiol is listed as 1, and salicylic acid is listed as 3, the weight ratio of cannabidiol to salicylic acid is 1:3 in the example or comparative example.

TABLE 1A

| Component | Example 1 | Example 2 | Example 3 |
|---|---|---|---|
| Cannabidiol | 1 | 1 | 1 |
| salicylic acid | 1 | 2 | 2.5 |

TABLE 1B

| | Compar- | Compar- | Compar- | Compar- | Compar- |
|---|---|---|---|---|---|
| | ative | ative | ative | ative | ative |
| Component | Example 1 | Example 2 | Example 3 | Example 4 | Example 5 |
| Cannabidiol | 1 | — | 2 | 3 | 1 |
| salicylic acid | — | 1 | 1 | 1 | 3.5 |

### EXPERIMENTAL EXAMPLES

### (1) Experimental Example 1: Cytotoxicity Evaluation of Examples 1 to 3 and Comparative Examples 3 to 5

A sebocyte cytotoxicity assay was performed on Examples 1 to 3 and Comparative Examples 3 to 5. The specific assay method is as follows:

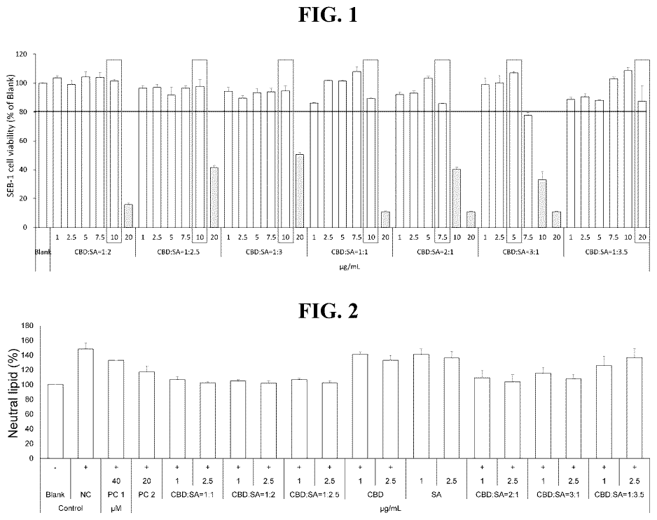

Sebocytes were seeded into a 96-well plate at a density of 1×10⁵ cells/well and cultured for 1 day, and after adding each of Examples 1 to 3 and Comparative Examples 3 to 5, further cultured for 24 hours. 3-(4,5-dimethylthiazol-2-yl)-2,5-diphenyltetrazolium bromide (MTT) was added to the plate at 2 mg/mL and allowed to react for 3 hours. The supernatant was discarded, formazan crystals generated by living cells were dissolved in dimethyl sulfoxide (DMSO) and then absorbance was measured at 570 nm to calculate cell viability. The results of the sebocyte cytotoxicity assay are shown in Table 2 and FIG. 1.

TABLE 2

| | Maximum |
|---|---|
| | concentration |
| Classification | (μg/mL) |
| Example 1 | 10 |
| Example 2 | 10 |
| Example 3 | 10 |
| Comparative Example 3 | 7.5 |
| Comparative Example 4 | 5 |
| Comparative Example 5 | 20 |

As a result of the assay, in Example 1 including cannabidiol and salicylic acid at a weight ratio of 1:1, Example 2 including cannabidiol and salicylic acid at a weight ratio of 1:2, Example 3 including cannabidiol and salicylic acid at a weight ratio of 1:2.5, and Comparative Example 5 including cannabidiol and salicylic acid at a weight ratio of 1:3.5, no cytotoxicity was exhibited at concentrations of 10 μg/mL or less.

On the other hand, in Comparative Example 3 including cannabidiol and salicylic acid at a weight ratio of 2:1, cytotoxicity was exhibited at concentrations greater than 7.5 μg/mL, and in Comparative Example 4 including cannabidiol and salicylic acid at 3:1, cytotoxicity was exhibited at concentrations greater than 5 μg/mL.

### (2) Experimental Example 2: Evaluation of Sebum Suppression Efficacy in Examples 1 to 3 and Comparative Examples 1 to 5

The results are shown in Table 3 and FIG. 2 by evaluating the sebum suppression efficacy in Examples 1 to 3 and Comparative Examples 1 to 5. Here, considering that cytotoxicity was observed at a concentration exceeding 5 μg/mL in Comparative Example 4 among the examples and the comparative examples regarding the mixture of cannabidiol and salicylic acid, the sebum suppression efficacy was evaluated at concentrations less than 5 μg/mL, which is a safe concentration in terms of cytotoxicity, for each example and comparative example.

The specific experimental method is as follows: Using a medium containing FBS and antibiotics, sebocytes were equally divided into 96-well plates at 1.0×10⁵ cells/well and cultured at 37° C. in a 5% CO₂ incubator for 24 hours. The cultured cells were treated with a mixture of the sample of each example and the medium at 37° C. in a 5% CO₂ incubator for 24 hours. Here, each sample had the same total amount but a different concentration. In addition, each sample was treated with 50 μM arachidonic acid, which is a causative factor that promotes sebum secretion, and allowed to react. The sample of each example was not added to a medium of a negative control, and lupeol and resveratrol were used as positive controls. Afterward, the amount of neutral lipid produced in each cell was measured and compared with the amount of neutral lipid in the negative control to calculate the sebum suppression efficacy. For example, provided that the amount of neutral lipid in the negative control is 100 and the amount of neutral lipid in the experimental group is 75, the sebum suppression efficacy is calculated as 25%. Acne skin secretes sebum with a higher neutral lipid content than normal skin, and it can be evaluated that the lower the measured amount of neutral lipid, the higher the sebum suppression efficacy.

TABLE 3

| Classification | Concentration | | Sebum suppression efficacy (%) |
|---|---|---|---|
| Lupeol | 40 | mM | 16 |
| Resveratrol | 20 | μg/mL | 32 |
| Example 1 | 1 | μg/mL | 28 |
| | 2.5 | μg/mL | 31 |
| Example 2 | 1 | μg/mL | 29 |
| | 2.5 | μg/mL | 31 |
| Example 3 | 1 | μg/mL | 28 |
| | 2.5 | μg/mL | 31 |
| Comparative | 1 | μg/mL | 8 |
| Example 1 | 2.5 | μg/mL | 16 |
| Comparative | 1 | μg/mL | 8 |
| Example 2 | 2.5 | μg/mL | 13 |
| Comparative | 1 | μg/mL | 26 |
| Example 3 | 2.5 | μg/mL | 30 |
| Comparative | 1 | μg/mL | 22 |
| Example 4 | 2.5 | μg/mL | 27 |
| Comparative | 1 | μg/mL | 15 |
| Example 5 | 2.5 | μg/mL | 8 |

The results show that, in Example 1 including cannabidiol and salicylic acid at a weight ratio of 1:1, Example 2 including cannabidiol and salicylic acid at a weight ratio of 1:2, and Example 3 including cannabidiol and salicylic acid at a weight ratio of 1:2.5, a notable sebum suppression efficacy of 28% or more at a concentration of 1 μg/mL and a notable sebum suppression efficacy of 31% or more at a concentration of 2.5 μg/mL were confirmed. In addition, it is confirmed that Examples 1 to 3 show a higher sebum suppression efficacy than a positive control lupeol, and a sebum suppression efficacy similar to resveratrol.

On the other hand, it can be confirmed that, in Comparative Example 1 including only cannabidiol, Comparative Example 2 including only salicylic acid, Comparative Example 3 including cannabidiol and salicylic acid at a weight ratio of 2:1, Comparative Example 4 including cannabidiol and salicylic acid at a weight ratio of 3:1, and Comparative Example 5 including cannabidiol and salicylic acid at a weight ratio of 1:3.5, the sebum suppression efficacy is insufficient at concentrations of 1 to 2.5 μg/mL compared to the examples.

According to embodiments of the invention, a composition for suppressing sebum that effectively suppresses sebum synthesis by including cannabidiol and salicylic acid can be provided.

In addition, according to embodiments of the invention, when being applied to the skin, the composition for suppressing sebum can improve inflammatory diseases such as acne, dermatitis, and the like.

The effects that can be obtained from embodiments of the invention are not limited to the effects mentioned above, and other effects that have not been mentioned will be clearly understood by those of ordinary skill in the art to which the invention belongs from the description below.

While specific parts of the invention have been described in detail above, it is clear to those skilled in the art that these specific parts are merely preferred embodiments, and the scope of the embodiment of the invention is not limited thereto. Thus, the substantial scope of the embodiment of the invention will be defined by the accompanying claims and their equivalents.

Although certain embodiments and implementations have been described herein, other embodiments and modifications will be apparent from this description. Accordingly, the inventive concepts are not limited to such embodiments, but rather to the broader scope of the appended claims and various obvious modifications and equivalent arrangements as would be apparent to a person of ordinary skill in the art.

What is claimed is:

1. A composition for suppressing sebum comprising:
cannabidiol and salicylic acid.

2. The composition of claim 1, wherein the weight ratio of the cannabidiol and the salicylic acid is 1:0.8 to 3.

3. The composition of claim 1, wherein the composition for suppressing sebum is present at a concentration of less than 5 μg/mL.

4. The composition of claim 1, wherein, at a concentration of 1 μg/mL, the composition for suppressing sebum reduces sebum production in sebocytes by 27% or more compared to a control containing 50 μM arachidonic acid, as measured by a lipogenesis assay.

5. The composition of claim 1, wherein, at a concentration of 2.5 μg/mL, the composition for suppressing sebum reduces sebum production in sebocytes by 30% or more compared to a control containing 50 μM arachidonic acid, as measured by a lipogenesis assay.

6. A cosmetic composition comprising the composition for suppressing sebum of claim 1.

7. The cosmetic composition of claim 6, wherein the weight ratio of the cannabidiol and the salicylic acid is 1:0.8 to 3.

8. The cosmetic composition of claim 6, wherein the composition for suppressing sebum is present at a concentration of less than 5 μg/mL.

9. The cosmetic composition of claim 6, wherein, at a concentration of 1 μg/mL, the composition for suppressing sebum reduces sebum production in sebocytes by 27% or more compared to a control containing 50 μM arachidonic acid, as measured by a lipogenesis assay.

10. The cosmetic composition of claim 6, wherein, at a concentration of 2.5 μg/mL, the composition for suppressing sebum reduces sebum production in sebocytes by 30% or more compared to a control containing 50 μM arachidonic acid, as measured by a lipogenesis assay.

11. An external composition for skin comprising the composition for suppressing sebum of claim 1.

12. The external composition of claim 11, wherein the weight ratio of the cannabidiol and the salicylic acid is 1:0.8 to 3.

13. The external composition of claim 11, wherein the composition for suppressing sebum is present at a concentration of less than 5 μg/mL.

14. The external composition of claim 11, wherein, at a concentration of 1 μg/mL, the composition for suppressing sebum reduces sebum production in sebocytes by 27% or more compared to a control containing 50 μM arachidonic acid, as measured by a lipogenesis assay.

15. The external composition of claim 11, wherein, at a concentration of 2.5 μg/mL, the composition for suppressing sebum reduces sebum production in sebocytes by 30% or more compared to a control containing 50 μM arachidonic acid, as measured by a lipogenesis assay.

16. A pharmaceutical composition for treating or preventing an inflammatory disease, comprising the composition for suppressing sebum of claim 1.

17. The pharmaceutical composition of claim 16, wherein the weight ratio of the cannabidiol and the salicylic acid is 1:0.8 to 3.

18. The pharmaceutical composition of claim 16, wherein the composition for suppressing sebum is present at a concentration of less than 5 μg/mL.

19. The pharmaceutical composition of claim 16, wherein, at a concentration of 1 μg/mL, the composition for suppressing sebum reduces sebum production in sebocytes by 27% or more compared to a control containing 50 μM arachidonic acid, as measured by a lipogenesis assay.

20. The pharmaceutical composition of claim 16, wherein, at a concentration of 2.5 μg/mL, the composition for suppressing sebum reduces sebum production in sebocytes by 30% or more compared to a control containing 50 μM arachidonic acid, as measured by a lipogenesis assay.
